(19) United States
(12) Patent Application Publication
(10) Pub. No.: US 2002/0131973 A1
(43) Pub. Date: Sep. 19, 2002

(54) CYTOTOXIC AGENTS

(76) Inventors: KENNETH DAWSON BAGSHAWE, LONDON (GB); PHILIP JOHN BURKE, LONDON (GB)

Correspondence Address: EDWIN V. MERKEL, NIXON PEABODY LLP, CLINTON SQUARE, P O BOX 31051, ROCHESTER, NY, 14603, US

(21) Appl. No.: 09/091,933

(22) PCT Filed: Dec. 27, 1996
(86) PCT No.: PCT/GB96/03254

(30) Foreign Application Priority Data
Feb. 2, 1996 (CA) 2,168,680

(51) Int. Cl. A61K 39/395; A61K 39/42; A61K 39/44; A61K 39/385; C07K 17/14; C07K 16/00; C12P 21/08; C08H 1/00; C07K 14/00; C07K 17/00; C07K 1/00

(52) U.S. Cl. 424/178.1; 424/182.1; 424/185.1; 424/192.1; 424/193.1; 424/195.11; 530/391.1; 530/391.7; 530/402

(57) ABSTRACT

A therapeutic system for destroying a target cell within a host having a vascular compartment, the system comprising: (a) a compound comprising a target cellspecific portion and a portion which will convert a selected substantially noncytotoxic substance into a cytotoxic substance, and (b) said substantially noncytotoxic substance, wherein at least the said portion of compound (a) capable of said conversion is, following administration to the host, internalised into said target cell. Preferably, the portion which converts said substantially noncytotoxic substance into a cytotoxic substance requires a factor which is present in sufficient concentration within the target cell for the said portion to effect conversion of said substantially noncytotoxic substance into a cytotoxic substance and which factor is not present in sufficient concentration within the blood of the vascular compartment for the said portion to effect said conversion.

This invention relates to a system for the targeted delivery of cytotoxic agents to selected cells. It relates particularly to immunoconjugates and to pro-drugs and to their use in combination for the delivery of cytotoxic drugs to selected cells.

A method of targeted drug delivery, often referred to as Antibody-Directed Enzyme Pro-drug Therapy (ADEPT), has been disclosed in WO88/07378. In this approach an enzyme, targeted to cancer cells via a monoclonal antibody fragment, is used to release a therapeutic drug from an independently administered pro-drug. This two-step approach has the potential to overcome many of the limitations of current chemotherapy strategies and to allow the generation of high intratumoural concentrations of drug whilst minimising damage to normal tissues. One enzyme can convert hundreds of pro-drug molecules every minute so this approach combines selective delivery with adequate dosage.

In order to achieve targeted delivery the conjugate is first injected or infused into the patient and allowed to localise at tumour cells. After a period of time to allow non-tumour bound conjugate to clear from the patient by metabolism and excretion the pro-drug is either injected or infused. Upon reaching the target cells the pro-drug is converted to a cytotoxic drug by the enzyme portion of the conjugate and the drug kills the cell to which the targeting portion is bound.

The ADEPT system is also described in relation to specific enzymes and pro-drugs in EP 0 302 473 and WO 91/11201 as well as in many journal articles including Knox & Connors (1995) Clin. Immunother. 3, 136-153 which notes that the targeted antibody-enzyme conjugate remains extracellular; Hellström et al (1991) Eur. J. Cancer 27, 1342-1343 which notes that the drug is preferentially released outside the cell where it can penetrate both into tumour cells that have bound the conjugate; Blakey et al (1995) Br. J. Cancer 72, 1083-1088 which notes that a small molecular weight cytotoxic drug is generated locally at the tumour site and outside the cell; Senter et al (1989) Cancer Res. 49, 5789-5792 which indicates that the ADEPT approach is designed to circumvent the need to deliver the cytotoxic agent inside the cell since the drug is extracellularly released by the enzyme; and Sedlacek et al (1992) Contributions to Oncology 43, 121-, Karger, Germany indicates that a pro-drug used in ADEPT has to be stable in serum and interstitium and should not diffuse into cells.

Friedlos and Knox (1992) Biochem. Pharm. 44, 631-635 and Knox et al (1993) Cancer Metastasis Rev. 12, 195-212 discuss reductive processes for activating pro-drugs and concludes that enzymes requiring reducing equivalents may be used to activate a pro-drug provided that the necessary co-factor is provided in the blood; NADPH was considered unsuitable as a source of reducing equivalents for the bioreductive activation of pro-drugs by a reductase enzyme in ADEPT.

A potential limitation of ADEPT is that a relatively low proportion of administered antibody-enzyme conjugate localises at tumour cells whereas a considerable amount persists in blood and normal tissues for several days. Although the concentration of enzyme localised at tumour cells is greater than in normal tissues the volume of the latter is much larger so, overall, the amount of conjugate in normal tissues is considerably in excess. The enzyme in this residual conjugate is able to convert pro-drug to drug causing toxicity to normal tissues and restricting the dose of pro-drug which can be administered. Such effects limit the usefulness of this delivery system. To minimise the amount of conjugate in blood and normal tissues methods for the inactivation and/or clearance of excess antibody-enzyme conjugate have been disclosed in WO 89/10140.

WO 93/13806 describes a further modification of the ADEPT system comprising a three component kit of parts for use in a method of destroying target cells in a host. The first component comprises a target cell-specific portion and an enzymatically active portion capable of converting a pro-drug into a cytotoxic drug; the second component is a pro-drug convertible by said enzymatically active portion to the cytotoxic drug; and the third component comprises a portion capable of at least partly restraining the component from leaving the vascular compartment of a host when said compound is administered to the vascular compartment, and an inactivating portion capable of converting the cytotoxic drug into a less toxic substance.

There remains, however, a continuing need to improve the level of specificity of ADEPT.

The present invention addresses the problem of pro-drug conversion by immunoconjugate not bound to target cells.

A first aspect of the invention provides a therapeutic system for destroying a target cell within a host having a vascular compartment, the system comprising:

(a) a compound comprising a target cell-specific portion and a portion which will convert a selected substantially non-cytotoxic substance into a cytotoxic substance; and

(b) said substantially non-cytotoxic substance

wherein at least the said portion of compound (a) capable of said conversion is, following administration to the host, internalised into said target cell.

The entity which is recognised by the target cell-specific portion may be any suitable entity which is expressed by tumour cells, virally-infected cells, pathogenic microorganisms, cells introduced as part of gene therapy or normal cells of the body which one wishes to destroy for a particular reason. The entity should preferably be present or accessible to the targeting portion in significantly greater concentrations in or on cells which are to be destroyed than in any normal tissues of the host that cannot be functionally replaced by other therapeutic means. Use of a target expressed by a cancer cell would not be precluded, for example, by its equal or greater expression on an endocrine tissue or organ. In a life-saving situation the organ could be sacrificed provided its function was either not essential to life, for example in the case of the testes, or could be supplied by hormone replacement therapy. Such considerations would apply, for instance, to the thyroid gland, parathyroids, adrenal cortex and ovaries.

The entity which is recognised will often be an antigen. Tumour-associated antigens, when they are expressed on the cell membrane or secreted into tumour extra-cellular fluid, lend themselves to the role of targets for antibodies.

The term “tumour” is to be understood as referring to all forms of neoplastic cell growth, including tumours of the lung, liver, blood cells (leukaemias), skin, pancreas, colon, prostate, uterus or breast.

It is preferred if the target cell, such as a tumour cell, is accessible to the compound of the invention via the vasculature.

The antigen-specific portion may be an entire antibody (usually, for convenience and specificity, a monoclonal antibody), a part or parts thereof (for example an Fab fragment or F(ab′)₂) or a synthetic antibody or part thereof. A conjugate comprising only part of an antibody may be advantageous by virtue of optimizing the rate of clearance from the blood and may be less likely to undergo non-specific binding due to the Fc part. Suitable monoclonal antibodies to selected antigens may be prepared by known techniques, for example those disclosed in “Monoclonal Antibodies: A manual of techniques”, H. Zola (CRC Press, 1988) and in “Monoclonal Hybridoma Antibodies: Techniques and Applications”, J. G. R. Hurrell (CRC Press, 1982). All references mentioned in this specification are incorporated herein by reference. Bispecific antibodies may be prepared by cell fusion, by reassociation of monovalent fragments or by chemical cross-linking of whole antibodies, with one part of the resulting bispecific antibody being directed to the cell-specific antigen and the other to the enzyme. The bispecific antibody can be administered bound to the enzyme or it can be administered first, followed by the enzyme. It is preferred that the bispecific antibodies are administered first, and after localization to the tumour cells, the enzyme is administered to be captured by the tumour localized antibody. Methods for preparing bispecific antibodies are disclosed in Corvalan et al (1987) Cancer Immunol. Immunother. 24, 127-132 and 133-137 and 138-143, and Gillsland et al (1988) Proc. Natl. Acad. Sci. USA 85, 7719-7723.

The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are linked via a flexible oligopeptide.

There are potential advantages of using antibody fragments, rather than whole antibodies. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites. Fragmentation of intact immunoglobulins to produce F(ab′)₂ fragments is disclosed by Harwood et al (1985) Eur. J. Cancer Clin. Oncol. 21, 1515-1522.

IgG class antibodies are preferred.

In general the target cell-specific portion should incorporate a targeting specificity and a cellular outer-membrane crossing facility. The target cell-specific component may achieve this function as a single entity (ie an internalising antibody) or it may require a combination of molecules ie a targeting-specific portion and an internalising-specific portion. Studies with monoclonal antibody-toxin immunoconjugates have demonstrated that such an approach is possible where a monoclonal antibody is used to target selected cells and ricin B chain, conjugated to the antibody, is used to internalise the conjugate into the cell and deliver a toxic moiety, often ricin A chain (E. J. Wawrzynczak (1991) B.J. Cancer, 64, 624-630).

This invention extends to any chemically or protein-engineered modification of a selected targeting agent which allows it to translocate into cells. In addition, chemical or genetic modifications to the delivery agent which allow the intracellular routing of the conjugate to specific regions of the cell eg cytosol, golgi apparatus or the nucleus.

It is desirable that the monoclonal antibody of this invention is of a type which is capable of internalisation by target cells. An example of an antibody of this type is the anti-μ antibody DA4-4 which is internalised by malignant human B lymphocytes (Geissler et al (1992) Cancer Research, 52, 2907-2915). A further example, of an antibody internalising to neuroblastoma cells, has been reported (Novak-Hofer, I, (1995) Cancer Research, 55, 46-50). An antibody which internalises in a number of human carcinomas (colon, breast, ovary and lung) is BR 96 (Hellstrom, I, et al (1990) Cancer Research, 50, 2183-2190). Internalising antibodies have also been described (Schumacher et al (1992) Nuclear medicine and biology, 19, 809-824).

The target cell-specific portion may be a ligand which is bound by cell-surface receptors and internalised by receptor-mediated endocytosis. Known ligands of this type are transferrin (Ciechanover et al (1983) J. Biol Chem, 258, 9681-9689), low density lipoprotein (Brown et al (1983) Cold Spring Harbour symp. Quant. Biol. 46, 713-734) and epidermal growth factor (Carpenter et al (1976) J. Cell Biol. 71, 159-171). It is preferable if the receptor is overexpressed on the target cells.

Suitably, the target cell-specific portion is a polymer or liposome with target cell-localising characteristics.

By “at least the said portion of compound (a) capable of said conversion is, following administration to the host, internalised into said target cell” we mean that once the compound has reached, and probably bound to, the target cell the whole compound, or at least the portion capable of said conversion, is taken into the cell.

Uptake into the cell can be by passive means or by active means. Preferably the uptake into the cell is active. More preferably the uptake of the compound of the invention is brought about by receptor-mediated endocytosis. In other words, the compound binds to a cell-surface receptor which internalises, carrying the compound with it.

It is readily determined using methods known in the art whether or not at least said portion of compound (a) capable of said conversion is internalised into said target cell. Using these known methods suitable compounds can be selected for use in the invention.

A particularly convenient method of determining whether a compound is internalised (and, in particular, whether at least said portion of compound (a) capable of said conversion is internalised) is to radiolabel the compound (preferably the radiolabel is present on the portion capable of said conversion), to incubate said radiolabelled compound with cells and to remove cell-surface bound compound after a suitable period of time. Radioactivity still associated with the cells is due to internalised compound (or at least said portion of said compound radioactively labelled). This method is described in Ypke et al (1992) Cancer Res. 52, 5291-5925, incorporated herein by reference. The cells are conveniently cells in culture in vitro. Suitably, the cells are cultured cells of a cell type which is a target cell when present in the host. For example, the cultured cell is suitably a cultured cancer cell. Cultured cancer cells are well known in the art.

Suitable periods of time and suitable amounts of compound are disclosed below.

Preferably this method is used to determine whether an antibody-enzyme conjugate of the invention is internalised.

Another particularly convenient method of determining whether a compound is internalised (and, in particular, whether at least said portion of compound (a) capable of said conversion is internalised) is to attach a fluorescent-labelled antibody to the compound under test, and to observe the location of the fluorescence in cells using a confocal microscope. This method of immunofluorescence microscopy is described in Garnett & Baldwin (1986) Eur. J. Cell Biol. 41, 2143-2211, incorporated herein by reference. As before, the cells are conveniently cells in culture in vitro. Suitably the cells are cultured cells of a cell type which is a target cell when present in the host. Preferably, this method is used to determine whether an antibody-enzyme conjugate of the invention is internalised.

Suitable periods of time and suitable amounts of compound are disclosed below.

Whether or not a particular compound will be internalised can be readily determined using one of the methods described above and therefore a suitable compound for use in the invention can be readily identified or selected.

Suitable compounds of the invention may be prepared from antibodies which bind to a cell-surface molecule which is internalised, for example a receptor molecule which is internalised. Antibodies raised to such cell-surface molecules are likely to be antibodies which are internalised by cells which have such molecules on their surface. Whether such antibodies are internalised by the cells can be determined using the methods described above. The antibodies can be converted into a compound of the invention by, for example, conjugating to a suitable enzyme.

Cell-surface molecule which can be internalised, including cell surface molecules which are overexpressed by tumour cells, are known in the art.

It will be appreciated that some antibodies are intrinsically capable of internalisation when they reach and bind to a target cell whereas other antibodies are not capable of internalisation. Examples of antibodies which internalise include C242 and 454A12 (Blakey et al (1994) Cell Biophysics, 24-25, 175-183); BR64 and BR96 (Hellstrom et al (1990) Cancer Research, 50, 2183-2190); anti-gp78 (Nabi et al (1992) Cancer Metastasis Rev. 11, 5-20); and 79IT/36 (Byers et al (1991) Cancer Research, 51, 1990-1995).

Examples of antibodies which do not internalise include A5B7 (Blakey et al (1994) Cell Biophysics, 24-25, 175-183); and L6 (Hellstrom et al (1990) Cancer Research, 50, 2183-2190). Both of these enzymes have been used previously in the ADEPT system.

By “internalisation” or “being internalised” we mean that a reasonable proportion of the compound that reaches and binds to the target cells is taken up into the cells in a reasonable period of time.

By a “reasonable proportion” we mean at least 5%, more preferably at least 10%, and still more preferably at least 20%, and in further preference 40% of the compound is taken up by the cells. By a “reasonable period of time” we mean within at least 12 hours, preferably within at least 2 hours, more preferably within at least 1 hour, and still more preferably within at least 30 minutes. Whether a compound is internalisable is conveniently determined in vitro for example, by using the methods described above.

It will be appreciated that internalised antibodies are usually degraded by intracellular enzymes so there is little advantage to be gained by longer exposure of the target cell to the antibody.

It is most preferred if more than 20% of the compound is taken up by the cells in less than 2 hours.

It will also be appreciated that for any selected cell type and selected compound internalisation can be determined in vitro as discussed above, and it is convenient to assess the internalisation of a particular compound using the in vitro methods disclosed. The in vitro determination of internalisation is a very good indicator of internalisation by an appropriate target cell in vitro. In assessing the internalisation of a compound of the invention in vitro it is convenient to carry out the methods disclosed above with between 1 and 10 million compounds of the invention per cell, more preferably between 10 and 100,000 and still more preferably between 100 and 10,000. It will be appreciate that for those compounds of the invention which bind selectively to a cell surface molecule prior to internalisation, the cell will become saturated with such compounds. Thus, it is convenient that in those circumstances the number of compounds of the invention per said cell surface molecule is between 1 and 100, more preferably between 1 and 20 and more preferably between 1 and 10.

It will be appreciated that as long as the portion capable of said conversion is internalised the benefits of the invention are obtained.

It is preferred if the target cell-specific portion comprises an intact antibody, or at least an antibody fragment comprising an Fc portion, because the Fc portion may aid internalisation.

It is less preferred if the target cell-specific portion comprises an antibody or fragment or derivative thereof.

Conveniently the portion capable of converting a substance into another substance is an enzyme (or at least is a macromolecule which has catalytic activity and could, therefore, be a catalytic RNA molecule or a catalytic carbohydrate molecule or at least the catalytic portion of an enzyme).

It is likely that the portion of the compound capable of converting a substance into another substance, when it is an enzymatically active portion, will be enzymatically active in isolation from the target cell-specific portion but it is necessary only for it to be enzymatically active when it is internalised and is present within the cell.

The two portions of the compound of the first aspect of the invention may be linked together by any of the conventional ways of cross-linking polypeptides, such as those generally described in O'Sullivan et al (1979) Anal. Biochem. 100, 100-108. For example, the antibody portion may be enriched with thiol groups and the enzyme portion reacted with a bifunctional agent capable of reacting with those thiol groups, for example the N-hydroxysuccinimide ester of iodoacetic acid (NHIA) or N-succinimidyl-3-(2-pyridyldithio)propionate (SPDP). Amide and thioether bonds, for example achieved with m-maleimidobenzoyl-N-hydroxysuccinimide ester, are generally more stable in vivo than disulphide bonds.

It may not be necessary for a whole enzyme to be present in the compound of the first aspect of the invention but, of course, the catalytic portion must be present.

Alternatively, the compound may be produced as a fusion compound by recombinant DNA techniques whereby a length of DNA comprises respective regions encoding the two portions of the compound of the invention either adjacent to one another or separated by a region encoding a linker peptide which does not destroy the desired properties of the compound. Conceivably, the two portions of the compound may overlap wholly or partly. The antibody component of the fusion must be represented by at least one binding site. Examples of the construction of antibody (or antibody fragment)-enzyme fusions are disclosed by Neuberger et al (1984) Nature 312, 604.

The DNA is then expressed in a suitable host to produce a polypeptide comprising the compound of this aspect of the invention. Thus, the DNA encoding the polypeptide constituting the compound of this aspect of the invention may be used in accordance with known techniques, appropriately modified in view of the teachings contained herein, to construct an expression vector, which is then used to transform an appropriate host cell for the expression and production of the polypeptide of the invention. Such techniques include those disclosed in U.S. Pat. Nos. 4,440,859 issued Apr. 3, 1984 to Rutter et al, 4,530,901 issued Jul. 23, 1985 to Weissman, 4,582,800 issued Apr. 15, 1986 to Crowl, 4,677,063 issued Jun. 30, 1987 to Mark et al, 4,678,751 issued Jul. 7, 1987 to Goeddel, 4,704,362 issued Nov. 3, 1987 to Itakura et al, 4,710,463 issued Dec. 1, 1987 to Murray, 4,757,006 issued Jul. 12, 1988 to Toole, Jr. et al, 4,766,075 issued Aug. 23, 1988 to Goeddel et al and 4,810,648 issued Mar. 7, 1989 to Stalker, all of which are incorporated herein by reference.

The DNA encoding the polypeptide constituting the compound of this aspect of the invention may be joined to a wide variety of other DNA sequences for introduction into an appropriate host. The companion DNA will depend upon the nature of the host, the manner of the introduction of the DNA into the host, and whether episomal maintenance or integration is desired.

Generally, the DNA is inserted into an expression vector, such as a plasmid, in proper orientation and correct reading frame for expression. If necessary, the DNA may be linked to the appropriate transcriptional and translational regulatory control nucleotide sequences recognised by the desired host, although such controls are generally available in the expression vector. The vector is then introduced into the host through standard techniques. Generally, not all of the hosts will be transformed by the vector. Therefore, it will be necessary to select for transformed host cells. One selection technique involves incorporating into the expression vector a DNA sequence, with any necessary control elements, that codes for a selectable trait in the transformed cell, such as antibiotic resistance. Alternatively, the gene for such selectable trait can be on another vector, which is used to co-transform the desired host cell.

Host cells that have been transformed by the recombinant DNA of the invention are then cultured for a sufficient time and under appropriate conditions known to those skilled in the art in view of the teachings disclosed herein to permit the expression of the polypeptide, which can then be recovered.

Many expression systems are known, including bacteria (for example E. coli and Bacillus subtilis), yeasts (for example Saccharomyces cerevisiae), filamentous fungi (for example Aspergillus), plant cells, animal cells and insect cells.

The vectors include a procaryotic replicon, such as the ColE1 ori, for propagation in a procaryote, even if the vector is to be used for expression in other, non-procaryotic, cell types. The vectors can also include an appropriate promoter such as a procaryotic promoter capable of directing the expression (transcription and translation) of the genes in a bacterial host cell, such as E. coli, transformed therewith.

A promoter is an expression control element formed by a DNA sequence that permits binding of RNA polymerase and transcription to occur. Promoter sequences compatible with exemplary bacterial hosts are typically provided in plasmid vectors containing convenient restriction sites for insertion of a DNA segment of the present invention.

Typical procaryotic vector plasmids are pUC18, pUC19, pBR322 and pBR329 available from Biorad Laboratories, (Richmond, Calif., USA) and pTrc99A and pKK223-3 available from Pharmacia, Piscataway, N.J., USA.

A typical mammalian cell vector plasmid is pSV_{L} available from Pharmacia, Piscataway, N.J., USA. This vector uses the SV40 late promoter to drive expression of cloned genes, the highest level of expression being found in T antigen-producing cells, such as COS-1 cells.

An example of an inducible mammalian expression vector is pMSG, also available from Pharmacia. This vector uses the glucocorticoid-inducible promoter of the mouse mammary tumour virus long terminal repeat to drive expression of the cloned gene.

Useful yeast plasmid vectors are pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers his3, trp1, leu2 and ura3. Plasmids pRS413-416 are Yeast Centromere plasmids (YCps).

A variety of methods have been developed to operatively link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion as described earlier, is treated with bacteriophage T4 DNA polymerase or E. coli DNA polymerase I, enzymes that remove protruding, 3′-single-stranded termini with their 3′-5′-exonucleolytic activities, and fill in recessed 3′-ends with their polymerizing activities.

The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of sources including International Biotechnologies Inc, New Haven, Conn., USA.

A desirable way to modify the DNA encoding the polypeptide of this aspect of the invention is to use the polymerase chain reaction as disclosed by Saiki et al (1988) Science 239, 487-491.

In this method the DNA to be enzymatically amplified is flanked by two specific oligonucleotide primers which themselves become incorporated into the amplified DNA. The said specific primers may contain restriction endonuclease recognition sites which can be used for cloning into expression vectors using methods known in the art.

Exemplary genera of yeast contemplated to be useful in the practice of the present invention are Pichia, Saccharomyces, Kluyveromyces, Candida, Torulopsis, Hansenula, Schizosaccharomyces, Citeromyces, Pachysolen, Debaromyces, Metschunikowia, Rhodosporidium, Leucosporidium, Botryoascus, Sporidiobolus, Endomycopsis, and the like. Preferred genera are those selected from the group consisting of Pichia, Saccharomyces, Kluyveromyces, Yarrowia and Hansenula. Examples of Saccharomyces are Saccharomyces cerevisiae, Saccharomyces italicus and Saccharomyces rouxii. Examples of Kluyveromyces are Kluyveromyces fragilis and Kluyveromyces lactis. Examples of Hansenula are Hansenula polymorpha, Hansenula anomala and Hansenula capsulata. Yarrowia lipolytica is an example of a suitable Yarrowia species.

Methods for the transformation of S. cerevisiae are taught generally in EP 251 744, EP 258 067 and WO 90/01063, all of which are incorporated herein by reference.

Suitable promoters for S. cerevisiae include those associated with the PGK1 gene, GAL1 or GAL10 genes, CYC1, PHO5, TRP1, ADH1, ADH2, the genes for glyceraldehyde-3-phosphate dehydrogenase, hexokinase, pyruvate decarboxylase, phosphofructokinase, triose phosphate isomerase, phosphoglucose isomerase, glucokinase, α-mating factor pheromone, a-mating factor pheromone, the PRB1 promoter, the GUT2 promoter, and hybrid promoters involving hybrids of parts of 5′ regulatory regions with parts of 5′ regulatory regions of other promoters or with upstream activation sites (eg the promoter of EP-A-258 067).

The transcription termination signal is preferably the 3′ flanking sequence of a eukaryotic gene which contains proper signals for transcription termination and polyadenylation. Suitable 3′ flanking sequences may, for example, be those of the gene naturally linked to the expression control sequence used, ie may correspond to the promoter. Alternatively, they may be different in which case the termination signal of the S. cerevisiae AHD1 gene is preferred.

It may not be necessary to use a conventional enzyme. Antibodies with catalytic capacity have been developed (Tramontano et al Science 234, 1566-1570) and are known as ‘abzymes’ or catalytic antibodies. These have the potential advantage of being able to be humanized to reduce their immunogenicity.

The selected substantially non-cytotoxic substance is conveniently a pro-drug and the cytotoxic substance is conveniently a cytotoxic drug.

In a particularly preferred embodiment the portion capable of converting a selected substantially non-cytotoxic substance into a cytotoxic substance requires a factor to effect the said conversion.

The factor is a factor which is present in sufficient concentration within the target cell for the said portion to effect conversion of said substantially non-cytotoxic substance into a cytotoxic substance and which factor is not present in sufficient concentration within the blood of the vascular component for the said portion to effect said conversion.

Thus, it will be readily seen that conversion of the selected substantially non-cytotoxic substance to the cytotoxic substance by the said portion of said compound will only be effected within the target cell to be killed.

It will be appreciated that any factor which is present in a sufficiently high concentration within the target cell (or at least within an appropriate compartment of the target cell) and in a sufficiently low concentration in the blood is suitable.

As mentioned above the portion capable of converting a substantially noncytotoxic substance into a cytotoxic substance is an enzyme.

Thus, conveniently the factor is a co-enzyme or another molecule which is required for the catalytic activity of the enzyme. Co-enzymes are known for many enzymes. Many oxidoreductases, dehydrogenases and reductases require NADH or NADPH as a supply of reducing equivalents (in one direction) or NAD⁺ or NADP⁺ as a sink for reducing equivalents (in the other direction). Thus, it is preferred if the enzyme requires NAD⁺, NADH, NADP⁺ or NADPH as a co-factor for the reaction catalysed. More preferably the enzyme is any one of an oxidase, dehydrogenase, reductase or diaphorase.

It is particularly preferred if the enzyme is a nitroreductase. Conveniently, the nitroreductase is the nitroreductase isolated from Walker 256 carcinoma cells shown to be an NAD(P)H dehydrogenase (quinone) classified as EC 1.6.99.2 (see Robertson et al (1986) J. Biol. Chem. 261, 15794-15799 and Knox et al (1988) Biochem. Pharmacol. 37, 4671-4677.

Other nitroreductases are also suitable such as those from Escherichia coli B described in Anlezark (1992) Biochem. Pharmacol. 44, 2289-2295. WO 93/08288 describes the nucleotide sequence of the E. coli B gene.

In a further preferred embodiment the substantially non-cytotoxic substance (b) is capable of entering the target cell.

Pro-drugs for this invention suitably are stable in plasma and preferably they should be lipophilic molecules capable of diffusion into cells or molecules which are capable of entering the cell by active transport.

A particularly preferred pro-drug is CB1954 (5-(aziridin-1-yl)-2,4-dinitrobenzamide) which is described in Cobb et al (1969) Biochem. Pharmacol. 18, 1519-1527. This pro-drug is activated by the Walker cell or the E. coli B nitroreductase but only in the presence of either NADH or NADPH.

For the purposes of tumour therapy it is desirable that the cytotoxic drug, once generated by the pro-drug/enzyme combination, is capable of entering and killing cells which have not been targeted by the conjugate. This “bystander effect” has been demonstrated in vitro with CB1954 and a co-culture of Walker cells and CHO cells. The latter, which do not contain nitroreductase were substantially killed (Knox et al (1988) Biochem. Pharmacol. 37, 4661-4669).

Other pro-drugs may be used instead of CB1954. A suitable example is the benzyloxycarbonyl derivative of actinomycin D which has been shown to be less toxic than the parent drug and release actinomycin D on treatment with the E. coli nitroreductase (Mauger et al (1994) J. Med. Chem 37, 3452-3458).

The E. coli B nitroreductase (Anlezark et al (1992) Biochem. Pharmacol. 44, 2289-2295) has been shown to reduce either or both nitro groups of the pro-drug CB1954 in the presence of NADH or NADPH to generate a potent cytotoxic agent.

It is known that the biogenic co-factors for reductases, NADH and NADPH, are rapidly oxidised and degraded by serum enzymes (Friedlos & Knox (1992) Biochem. Pharmacol. 44, 631-635) so have a very short half-life. This situation will favour a targeting system in which a nitroreductase enzyme is delivered intracellularly to activate a pro-drug. There should be insufficient co-factor present in serum or normal tissues to allow enzyme not bound to target cells to activate the pro-drug.

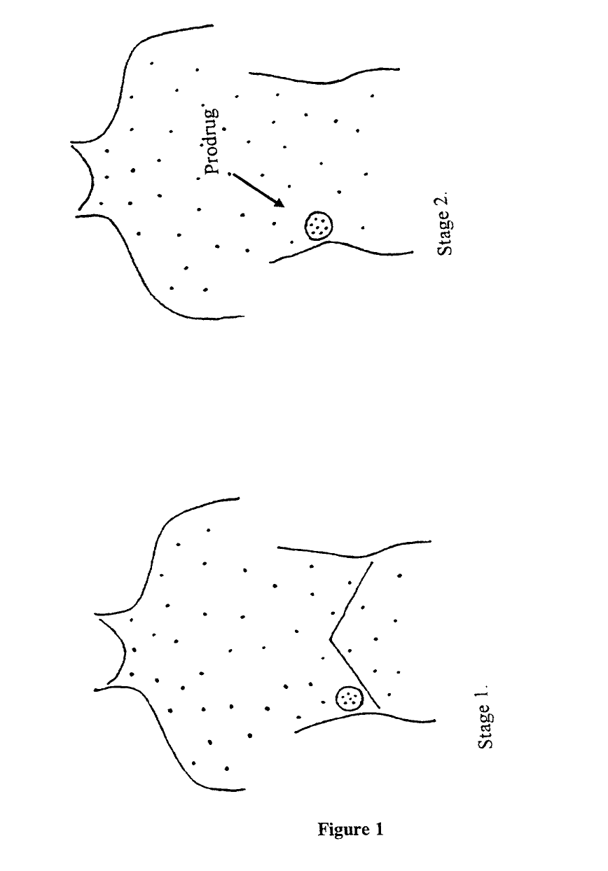

This invention therefore provides a particular conjugate which comprises a delivery component and an enzyme which requires a co-factor to function, eg a nitroreductase of the type described above. In this targeting system the delivery component binds to the cell and is internalized. The attached enzyme is taken by the delivery component into the cell where it is able to function using indigenous co-factor. The concept is illustrated in FIG. 1.

This approach has important advantages over a targeting system in which the conjugate is localised on the exterior of the cell surface or in the extracellular fluid surrounding the target cells. In the present invention unbound conjugate in blood or normal tissues will not be capable of activating pro-drug as no co-factor is accessible at these sites. Toxicity will be considerably reduced and the maximum dose of pro-drug may be administered. In addition, this invention will obviate the need for a clearance/inactivation system, which will simplify the treatment and greatly reduce its cost.

As with conventional ADEPT a low concentration of enzyme can release a large number of drug molecules from a suitable pro-drug. This catalytic component provides a significant advantage over previous attempts to internalise drugs or toxins by attachment to monoclonal antibodies.

It will be appreciated that it is preferred that the said factor which is present in sufficient concentration within the target cell for said portion to effect conversion does not form part of the system.

The compounds of the invention will be useful in the treatment of cancer. Conjugate will be administered and, after a suitable time period to allow maximum localisation at the tumour, pro-drug will be administered. Dosage will be determined by individual clinicians for individual patients.

Thus, a second aspect of the invention provides a method of destroying a target cell in a host, said host having a vascular compartment, the method comprising administering to the host

(a) a compound comprising a target cell-specific portion and a portion which will convert a selected substantially non-cytotoxic substance into a cytotoxic substance; and

(b)) said substantially non-cytotoxic substance

wherein at least the portion of compound (a) capable of said conversion is, following administration to the host, internalised into said target cell.

Preferably the target cell is a tumour cell. Preferably the host is a mammal.

A third aspect of the invention provides a method of treating a mammal harbouring a tumour, the method comprising the steps of administering to the mammal

(a) a compound comprising a target cell-specific portion and a portion which will convert a selected substantially non-cytotoxic substance into a cytotoxic substance; and

(b) said substantially non-cytotoxic substance

wherein at least the portion of compound (a) capable of said conversion is, following administration to the mammal, internalised into said target cell.

By “mammal” we include a human patient.

It will be appreciated that it is preferred that the said factor which is present in sufficient concentration within the target cell for said portion to effect conversion is not administered to the host or mammal.

A fourth aspect of the invention provides a compound comprising a target cell-specific portion and a portion which will convert a selected substantially non-cytotoxic substance into a cytotoxic substance wherein at least the portion of said compound capable of said conversion is, following administration to a suitable host, internalised into said target cell.

A fifth aspect of the invention provides a compound of the fourth aspect of the invention and a pharmaceutically acceptable carrier.

A sixth aspect of the invention provides a compound of the fourth aspect of the invention for use in medicine.

In the second and third aspects of the invention the components can be administered in any suitable way, usually parenterally, for example intravenously, intraperitoneally or intravesically, in standard, sterile, non-pyrogenic formulations of diluents and carriers, for example isolonic saline (when administered intravenously).

The formulations may conveniently be presented in unit dosage form and may be prepared by any of the methods well known in the art of pharmacy. Such methods include the step of bringing into association the active ingredient (compound of the invention) with the carrier which constitutes one or more accessory ingredients. In general the formulations are prepared by uniformly and intimately bringing into association the active ingredient with liquid carriers.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question.

The administration of the compound of the invention will be the similar to other ADEPT regimes. Suitably, it will be administered intravenously or by another appropriate route. The amount of compound is conveniently determined by the physician but his intention will be to maximise the amount administered.

After a suitable period of time (the time may be determined by the physician, but preferably within 12 hours) to allow the conjugate to localise at the tumour, the pro-drug is administered. This will be administered by standard routes for drug administration.

However, one difference between this system and previous ADEPT systems is that since the compound inside the tumour cells will be degraded by enzymes within the cell at a relatively fast rate, it may be desirable to administer the pro-drug either immediately before, at the same time as, or immediately after the conjugate.

The invention will now be described with reference to the following Examples and Figures wherein:

FIG. 1 shows the operational principle of IADEPT (internalised antibody-enzyme pro-drug therapy. AEC is antibody-enzyme conjugate. Stage 1: AEC localises at cancer site; residual AEC in normal tissue. Stage 2: Prodrug is administered; this can only be converted to drug by the action of enzyme inside cells.

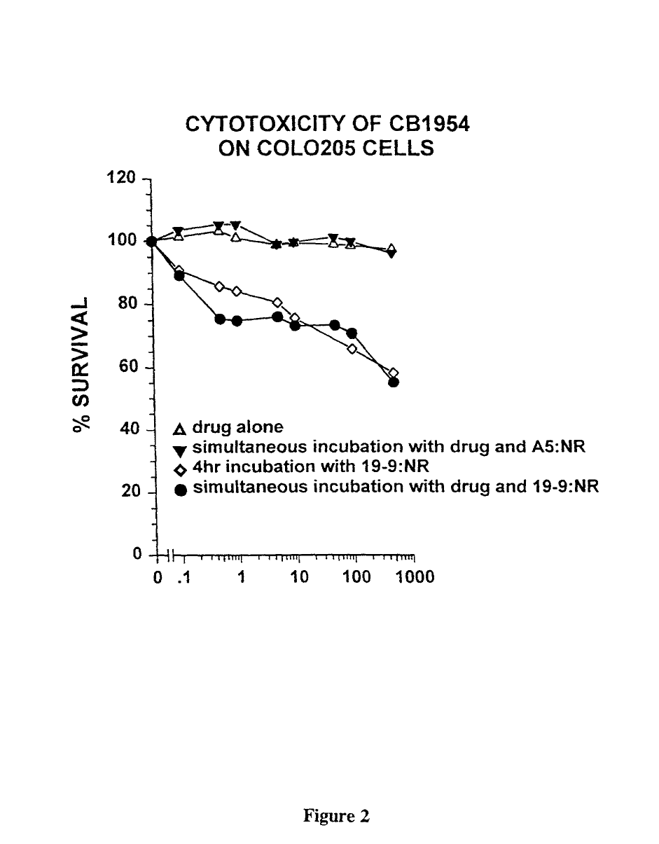

FIG. 2 shows the effect of non-internalising and internalising conjugates on the cytotoxicity of COLO-205 cells in the presence of CB1954.

### EXAMPLE 1

### Comparing Cytotoxicity of CB1954 With Internalising and Non-Internalising Antibodies

FIG. 2 shows the results of an experiment in which CB1954 was incubated with COLO-205 cells which had been pretreated with a conjugate of E. coli nitroreductase and 19-9, a monoclonal antibody known to internalise in this cell system. The cell kill observed is clearly due to intracellular activation of the CB1954. Use of a conjugate (A5NR) which did not internalise under these conditions gave no cell kill.

The result is particularly significant as antibody 19-9 is known to internalise very poorly into COLO cells (16% in 24 hours) and CB1954 is known to be a poor substrate for the enzyme. Very little active drug may therefore have been present in the COLO cells.

Preparation of 19.9-NR conjugate.

Conjugation was performed in phosphate-buffered saline (PBS) at room temperature. 50 mg 19.9 antibody in 13 ml PBS were reacted with a 12-fold molar excess of SMPB(4-(p-maleimidophenyl)-butyric acid N-hydroxysuccinimide ester) dissolved in a minimum volume of DMSO (approximately 50 μl) to insert maleimide groups. At the same time, sufficient pure E. coli B nitroreductase (16 mg in 5 ml of PBS) to give a 2-fold molar excess of enzyme over antibody was reacted with a 10-fold molar excess of 2-iminothiolane (dissolved in a minimum volume of DMSO) to introduce free thiol groups into the enzyme molecule. These activation reactions were allowed to proceed at room temperature for 1 hour, after which time both solutions were buffer exchanged into PBS to remove excess reagents, mixed together and left at room temperature for a further hour. The protein concentration of the conjugation mixture did not exceed 2 mg/ml. After this time an aliquot was taken for SDS-PAGE (4-15% gradient Phastgel, not reduced) to check for satisfactory formation of conjugate. Excess free thiol groups were blocked by the addition of approximately 5 mg of N-ethyl maleimide to the conjugation mixture and the mixture left to stand at room temperature with stirring for 1 hour. Glycine (1 mg/ml) was added before the mixture was concentration, using an Amicon stirred cell concentration with a PM10 membrane, to approximately 10 mg protein/ml. Conjugate was separated from free antibody and enzyme by gel filtration chromatography on a Superdex 200 16/60 column equilibrated in PBS. Fractions containing conjugate were concentrated and enzyme activity assessed by HPLC using the pro-drug bischloroethyl 2,4-dinitrocarboxamide (SN 23862) as substrate and also spectrophotometrically using menadione as substrate. Homogeneity was assessed by SDS-PAGE (4-15% gradient Phastgel, not reduced). Yield of conjugate was 35-40% and consisted of a mixture of 1:1 and 2:1 enzyme: antibody forms in approximately equal proportions together with smaller proportions of higher molecular weight conjugates and free antibody (<10% of the total yield).

Cytotoxicity assay

Colo205 cells were grown in suspension culture in RPMI 1640 medium supplemented with 10% heat-inactivated foetal calf serum, 2 mM L-glutamine, 50 IU/ml penicillin and 50 μg/ml streptomycin. Cultures were incubated at 37° C. in a humidified atmosphere with 5% CO₂ in air. For the in vitro cytotoxicity assay, 2 groups of cells in suspension at 10,000/ml were prepared in 10 ml plastic sterile test tubes. One group of cells were incubated with 0.05U/ml of 19/9-NR, and graded concentrations of CB1954 in triplicate at 37° C. for 2 hours. The second group of cells were incubated with 0.05U/ml of A5-NR and graded concentrations of CB1954 in triplicate at 37° C. for 2 hours. The cells were then spun at 1500 rpm for 5 min. Supernatant was decanted and the cell pellet resuspended in 1 ml of drug-free medium and the suspension incubated at 37° C. for 7 days. At the end of this incubation time, growth was determined by counting the cells in a coulter counter.

1. A therapeutic system for destroying a target cell within a host having a vascular compartment, the system comprising:
(a) a compound comprising a target cell-specific portion and a portion which will convert a selected substantially non-cytotoxic substance into a cytotoxic substance; and
(b) said substantially non-cytotoxic substance which is capable of entering the target cell
wherein at least the said portion of compound (a) capable of said conversion is, following administration to the host, internalised into said target cell, and wherein said portion which converts said substantially non-cytotoxic substance into a cytotoxic substance requires a factor which is present in sufficient concentration within the target cell for the said portion to effect conversion of said substantially non-cytotoxic substance into a cytotoxic substance and which factor is not present in sufficient concentration within the blood of the vascular compartment for the said portion to effect said conversion.

2. A system according to claim 1 wherein said portion which converts the substantially non-cytotoxic substance into a cytotoxic substance is an enzyme, or another macromolecule with catalytic activity.

3. A system according to claim 1 or 2 wherein the target cell-specific portion comprises an antibody or fragment or derivative thereof.

4. A system according to claim 3 wherein the antibody is the monoclonal antibody 19-9, anti-μ antibody DA4-4, or antibody BR96.

5. A system according to any one of claims 1 to 4 wherein the factor is an enzyme co-factor.

6. A system according to any one of claims 2 to 5 wherein the enzyme is a reductase, oxidase, dehydrogenase or diaphorase.

7. A system according to claim 5 wherein the enzyme co-factor is a nicotinamide co-factor.

8. A system according to claim 6 or 7 wherein the enzyme is a nitroreductase and the co-factor is NADH or NADPH.

9. A system according to claim 8 wherein the substantially non-cytotoxic substance is any one of CB1954 and actinomycin D pro-drug.

10. A method of destroying a target cell in a host, said host having a vascular compartment, the method comprising administering to the host
(a) a compound comprising a target cell-specific portion and a portion which will convert a selected substantially non-cytotoxic substance into a cytotoxic substance; and
(b) said substantially non-cytotoxic substance which is capable of entering the target cell
wherein at least the portion of compound (a) capable of said conversion is, following administration to the host, internalised into said target cell and wherein said portion which converts said substantially non-cytotoxic substance into a cytotoxic substance requires a factor which is present in sufficient concentration within the target cell for the said portion to effect conversion of said substantially non-cytotoxic substance into a cytotoxic substance and which factor is not present in sufficient concentration within the blood of the vascular compartment for the said portion to effect said conversion.

11. A method of treating a mammal harbouring a tumour, the method comprising the steps of administering to the mammal
(a) a compound comprising a target cell-specific portion and a portion which will convert a selected substantially non-cytotoxic substance into a cytotoxic substance; and
(b) said substantially non-cytotoxic substance which is capable of entering the target cell
wherein at least the portion of compound (a) capable of said conversion is, following administration to the mammal, internalised into said target cell and wherein said portion which converts said substantially non-cytotoxic substance into a cytotoxic substance requires a factor which is present in sufficient concentration within the target cell for the said portion to effect conversion of said substantially non-cytotoxic substance into a cytotoxic substance and which factor is not present in sufficient concentration within the blood of the vascular compartment for the said portion to effect said conversion.

12. A system according to claims 10 or 11 wherein said portion which converts the substantially non-cytotoxic substance into a cytotoxic substance is an enzyme, or another macromolecule with catalytic activity.

13. A system according to any one of claims 10 to 12 wherein the target cell-specific portion comprises an antibody or fragment or derivative thereof.

14. A method according to claim 13 wherein the antibody is the monoclonal antibody 19-9, anti-μ antibody DA4-4 or BR96.

15. A method according to any one of claims 10 to 14 wherein the factor is an enzyme co-factor.

16. A method according to any one of claims 12 to 15 wherein the enzyme is a reductase, oxidase, dehydrogenase or diaphorase.

17. A method according to claim 15 wherein the enzyme co-factor is a nicotinamide co-factor.

18. A method according to claim 16 or 17 wherein the enzyme is a nitroreductase and the co-factor is NADH or NADPH.

19. A method according to claim 18 wherein the substantially non-cytotoxic substance is any one of CB 1954 and actinomycin pro-drug.
